(19) United States
(12) Patent Application Publication
(10) Pub. No.: US 2026/0315524 A1
(43) Pub. Date: Oct. 15, 2026

(54) Rehydrating Composition of Electrolytes with Postbiotic and Zinc

(71) Applicant: Halewood Laboratories Pvt. Ltd., Ahmedabad, Gujarat (IN)

(72) Inventor: Krutin Kaushik CHATURVEDI, Ahmedabad, Gujarat (IN)

(21) Appl. No.: 19/117,260

(22) PCT Filed: Oct. 7, 2023
(86) PCT No.: PCT/IN2023/050909

§ 371 Date: Mar. 31, 2025

(30) Foreign Application Priority Data
Oct. 7, 2022 (IN) 202221057465

(51) Int. Cl. A61K 33/30; A61K 9/00; A61K 9/08; A61K 33/00; A61K 33/14; A61K 35/747; A61K 47/26

(52) U.S. Cl. CPC A61K 33/30; A61K 9/0053; A61K 9/08; A61K 33/00; A61K 33/14; A61K 35/747; A61K 47/26

(57) ABSTRACT

The present invention is in field of oral rehydration systems. More particularly, the present invention discloses rehydrating composition containing electrolytes along with postbiotic and zinc.

### FIELD OF INVENTION

The present invention is in field of oral rehydration systems. More particularly, the present invention relates to rehydrating composition of electrolytes along with post biotic and zinc.

### BACKGROUND OF INVENTION

Almost 1.6 million people die of diarrheal diseases globally, diarrhoea is the condition of having watery bowel movements throughout the day. The watery bowls lead to loss of vital electrolytes from the body which results in dehydration and Electrolyte imbalance. Generally, an ORS is recommended to maintain an electrolyte balance. ORS is a simple treatment for dehydration caused by diarrhoea, cholera, too much exposure to heat etc. It is used around the world and specifically in the developing nations where it saves lives of children suffering from dehydration due to diarrhoea, second leading cause of death amongst children.

Current WHO recommendations state that clinical management of acute diarrhoea should include administration of fluid and electrolytes along with zinc to replenish losses, it is been advised to administer along with nutritional support for restoration of intestinal microflora. In that case postbiotic is best solution along with electrolytes and zinc. It has a significant impact on program of treatment and prevention of acute diarrhoea. Postbiotic is a by-product of the fermentation process carried out by probiotics in the intestine.

Zinc, however, with the product has an important role in recovery from the symptoms of diarrhoea. The use of zinc with oral rehydration solutions has been shown to affect positive impacts by reducing the duration and severity of diarrheal episodes. Overall product gives an additive effect in the treatment of diarrhoea by maintaining mucosal integrity and improving electrolyte balance. But all 3 products ORS, Zinc and Postbiotics are available separately in market so it is not convenient for administration. According to available evidences, post-biotics do not need the viability in comparison with their parent cells and they exert health effects like live probiotic cells. Also, their preparation and application in various delivery systems is affordable, as discussed in article “Evaluating the Role of Postbiotics as a New Generation of Probiotics in Health and Diseases” by Homayouni-rad et al.

Therefore, it has been the effort of current inventors to device a formulation which combines the benefits of ORS, Zinc and Post Biotics.

### OBJECT OF THE INVENTION

In view of the above, the object of the invention is to provide a rehydrating composition of electrolytes, zinc and post biotic for diarrhoea and restoring the gut microbial flora.

### SUMMARY OF THE INVENTION

In an aspect the present invention provides a rehydrating composition for diarrhoea and restoring the gut microbial flora.

The rehydrating composition contains a combination of electrolytes, Zinc and Post Biotics.

In an aspect, the present invention provides a rehydrating composition comprising;
i. The ORS containing the Electrolytes, selected from, but not limited to, Sodium Chloride, Potassium Chloride, Sodium Citrate or combinations thereof, in an amount ranging between 0.2 to 0.7 μm/100 ml;
ii. Carbohydrates selected from glucose, dextrose, fructose, sucrose, maltodextrin and the like in an amount ranging between 1.2 to 2.5 μm/100 ml;
iii. Zinc salts equivalent to zinc in an amount ranging between 2 to 6 mg/100 ml; and
iv. Post biotics in an amount ranging between 25 mg-30 mg/100 ml.

The rehydrating composition of the present invention may comprise at least one ingredient selected from the group consisting of flavorants, colorants, vitamins, sweeteners, dietary supplements and the like.

The post biotic Immuno-LP20 is selected from heat killed Lactobacillus plantarum L-137 (HK L-137), which is not viable when administered, however, after reaching the intestine, these heat killed cells release bacterial components with key immune modulating effects and also have antagonizing properties against pathogens.

The zinc salts in the rehydrating composition of the present invention equivalent to the elemental zinc is selected from zinc sulfate, zinc sulfate monohydrate, zinc gluconate, zinc chloride, zinc citrate, zinc bicarbonate, zinc carbonate, zinc hydroxide, zinc lactate, zinc acetate, zinc fluoride, zinc bromide, and zinc sulfonate.

In an aspect, the rehydrating composition of the present invention is preservative free.

In another aspect, the pH of the rehydrating composition is in the range of 4.20 to 5.0 and the Osmolarity is 245 mOsmol/lit.

In yet another aspect, the rehydrating composition of the present invention is ready to drink composition.

In another aspect, the present invention provides a process for manufacturing the rehydrating composition as depicted in FIG. 1.

### DESCRIPTION OF THE FIGURE

FIG. 1: Depict the manufacturing process of the rehydrating composition.

### DETAILED DESCRIPTION OF THE INVENTION

In order to provide a clear and consistent understanding of the terms used in the present specification, a number of definitions are provided below. Moreover, unless defined otherwise, all technical and scientific terms as used herein have the same meaning as commonly understood to one of ordinary skill in the art to which this invention pertains.

The use of the word “a” or “an” when used in conjunction with the term “comprising” in the claims and/or the specification may not only mean “one”, but also encompasses the meaning of “one or more”, “at least one”, and “one or more than one”. Similarly, the word “another” may mean at least a second or more.

As used in this specification and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

Described herein is a rehydrating composition for diarrhoea and restoring the gut microbial flora.

In an embodiment, the rehydrating composition of the invention comprises a synergistic combination of electrolytes, Zinc and Post Biotics masked with delicious flavour which is suitable for the administration to all age groups including young children. Accordingly, the rehydrating composition of the invention comprises a combination of electrolytes with Zinc in a liquid ready to drink form in contrast to traditional dosage form of taking Zinc tablet supplementation additionally along with ORS.

In accordance with the above, the present invention relates to a rehydrating composition comprising;
i. The ORS containing the electrolytes, selected from, but not limited to, Sodium Chloride, Potassium Chloride, Sodium Citrate or combinations thereof, in an amount ranging between 0.2 to 0.7 μm/100 ml;
ii. Carbohydrates selected from glucose, dextrose, fructose; sucrose, maltodextrin and the like in an amount ranging between 1.2 to 2.5 μm/100 ml;
iii. Zinc salts equivalent to zinc in an amount ranging between 2 to 6 mg/100 ml; and
iv. Post biotics in an amount ranging between 25 mg to 30 mg/100 ml.

The rehydrating composition of the present invention comprise of excipients selected from the group consisting of flavorants, colorants, vitamins, dietary supplements, sweeteners and the like in appropriate amount. The flavors mask the salty taste of the rehydration composition and enhance the palatability. The sweeteners may compliment the flavours used in the present composition and May include natural or artificial sweeteners.

The electrolytes of the ORS (Oral rehydrating solution) used in the present composition are as per the World Health Organisation (WHO) guidelines.

The carbohydrates used in the present composition are in anhydrous form.

The post biotic Immuno-LP20 is selected from heat killed Lactobacillus plantarum L-137 (HK L-137), which is not viable when administered, however, after reaching the intestine, these heat killed cells release bacterial components with key immune modulating effects and also have antagonizing properties against pathogens. The advantage of using postbiotics against probiotics is that Post biotics helps in preventing recurrence of diarrhoea and maintains the gut flora. On the contrary, the probiotics are live cultures, majority of these live bacteria are destroyed by gastric juices, when consumed, and do not reach the intestine to give the desired effect.

The zinc salts in the rehydrating composition of the present invention equivalent to the elemental zinc is selected from zinc sulfate, zinc sulfate monohydrate, zinc gluconate, zinc chloride, zinc citrate, zinc bicarbonate, zinc carbonate, zinc hydroxide, zinc lactate, zinc acetate, zinc fluoride, zinc bromide, and zinc sufonate.

In the preferred embodiment, the composition comprises zinc sulphate monohydrate as a source of elemental zinc. Zinc is essential mineral that helps in maintaining normal physiological functions of the body. ORS with zinc is widely recommended by UNICEF, WHO for treatment of diarrhoea.

In another embodiment, the rehydrating composition of the present invention is preservative free.

In another embodiment, the pH of the rehydrating composition is in the range of 4.20 to 5.0 and the Osmolarity is 245 mOsmol/lit.

In yet another embodiment, the rehydrating composition of the present invention is ready to drink (RTI) composition.

In a preferred embodiment, the present invention discloses a rehydrating composition comprising;
i. The ORS containing the Electrolytes, selected from Sodium Chloride in an amount of 0.26 g/100 ml, Potassium Chloride in an amount of 0.15 μm/100 ml, Sodium Citrate in an amount of 0.29 μm/100 ml;
ii. Anhydrous dextrose in an amount of 1.35 μm/100 ml;
iii. Zinc sulfate monohydrate equivalent to zinc in an amount of 4 mg/100 ml; and
iv. Immuno-LP20 as Postbiotic in an amount of 25.0 mg/100 ml.

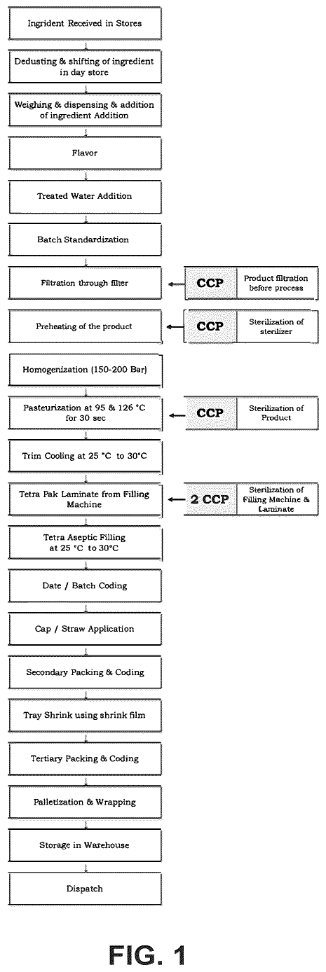

In yet another embodiment, the rehydrating composition of the present invention is manufactured by the process known to a person skilled in the art. As a general procedure depicted in FIG. 1, all the ingredients may be dry blended together; dispersed in water with agitated; and optionally heated to the appropriate temperature to dissolve all the constituents. The rehydrating composition is then packaged and sterilized to food grade standards as is known in the art.

One important factor driving interest in ready to drink rehydrating composition with post biotics and zinc is their inherent stability, both during industrial processes and storage. Maintaining stability of this product is a technological challenge as liquid ORS is already a very delicate product though post biotics carries dead microorganisms.

Accordingly, the rehydrating composition of the present invention is packed in 6-layered aseptic tetra packs. The rehydrating composition is stable for a period of 18 months without any added preservative in a ready to drink form.

In another embodiment, the present invention relates to the rehydrating composition for treating diarrhoea and restoring the gut microbial flora.

In yet another embodiment, the present invention relates to a method of treating diarrhoea and restoring the gut microbial flora comprising administering the rehydrating composition of the present invention to the subject in need.

In another preferred embodiment, the present invention disclose the ready to drink rehydrating composition packed in 6 layered aseptic tetra pack wherein one tetrapack of 200 ml provides 4 mg of Zinc, 0.52 g of NaCl, 0.3 g of KCl, 0.58 g Sodium Citrate and 2.7 g of Dextrose which is same as per recommendation of WHO.

In an advantageous embodiment;
The rehydrating composition of the present invention provides exact dosage and Osmolarity of ORS and Zinc which is recommended by WHO through combikit.
The rehydrating composition of one tetrapack of 200 ml according to the present invention provides 4 mg of Zinc, 0.52 g of NaCl, 0.3 g of KCl, 0.58 g Sodium Citrate and 2.7 g of Dextrose which is same as per recommendation of WHO.
Postbiotics used in the rehydrating composition helps in preventing recurrence of diarrhoea and maintains the gut flora.
The rehydrating composition provided in ready to drink liquid form prevents contamination which is generally seen while using contaminated water along with powder ORS that might cause some serious infections specially for infants.
The rehydrating composition as provided in the present invention has delicious fruit flavour, and easier to carry and thus, suitable for infants and children.
No chance of over dosage or low dose.

In yet another advantageous embodiment, the rehydrating composition of the invention does not require any cold storage or cold chain. The composition can be stored at cool temperatures.

### EXAMPLE

Examples are set forth to illustrate the nature of the invention and the manner of carrying it out. However, the invention should not be considered as being limited to the details thereof.

### Example 1: Composition

Each pack of 200 ml contains:—

| | |
|---|---|
| Components | Amount |

| | | |
|---|---|---|
| Sodium Chloride | 0.52 | gm |
| Potassium Chloride | 0.30 | gm |
| Sodium Citrate IP | 0.58 | gm |
| Dextrose Anhydrous | 2.70 | gm |
| Zinc Sulfate Monohydrate Eq. to Elemental Zinc | 4 | mg |
| Immuno-LP20 (Postbiotic) | 50 | mg |

| | |
|---|---|
| Orange flavour | Q.S |
| Excipients:- citric acid as acidity regulator | Q.S |
| Excipients: Sucralose as taste enhancer | Q.S |
| Water | Q.S |

### Example 2: Composition

Each pack of 200 ml contains:—

| | |
|---|---|
| Components | Amount |

| | | |
|---|---|---|
| Sodium Chloride | 0.52 | gm |
| Potassium Chloride | 0.30 | gm |
| Sodium Citrate IP | 0.58 | gm |
| Dextrose Anhydrous | 2.70 | gm |
| Zinc Sulfate Monohydrate Eq. to Elemental Zinc | 4 | mg |
| Immuno-LP20 (Postbiotic) | 50 | mg |

| | |
|---|---|
| Apple flavour | Q.S |
| Excipients:- citric acid as acidity regulator | Q.S |
| Excipients: Sucralose as taste enhancer | Q.S |
| Water | Q.S |

### Example 3: Process Development

All API's were mixed with ingredients in ratio of 1:1 or equal quantities, and were tested to check out for incompatibilities with respective API's or with any of its ingredients as depicted in FIG. 1.

### Example 4: Stability Studies

Conclusion: The product was stable and have complete efficacy.

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

1. A rehydrating composition comprising:
i. an oral rehydrating solution (ORS) containing at least one-electrolytes, comprising sodium chloride, potassium chloride, sodium citrate or a combinations thereof, in an amount ranging between 0.2 to 0.7 μm/100 ml;
ii. a carbohydrate comprising glucose, dextrose, fructose, sucrose, maltodextrin, or a combination thereof in an amount ranging between 1.2 to 2.5 μm/−100 ml;
iii. a zinc salts comprising zinc in an amount ranging between 2 to 6 mg/100 ml; and
iv. a post biotic in an amount ranging between 25 to 30 mg/100 ml.

2. The rehydrating composition as claimed in claim 1, wherein the composition further comprises an excipients, the excipient comprising a flavorant, a pH adjuster, a colorant, a vitamin, a dietary supplement, a sweetener or a combination thereof.

3. The rehydrating composition as claimed in claim 1, wherein the post-biotic is selected from heat killed Lactobacillus plantarum L-137 (HK L-137).

4. The rehydrating composition as claimed in claim 1, wherein the zinc salts comprises zinc sulfate, zinc sulfate monohydrate, zinc gluconate, zinc chloride, zinc citrate, zinc bicarbonate, zinc carbonate, zinc hydroxide, zinc lactate, zinc acetate, zinc fluoride, zinc bromide, zinc sufonate, or a combination thereof.

5. The rehydrating composition as claimed in claim 1, wherein the zinc salts is zinc sulfate monohydrate.

6. The rehydrating composition as claimed in claim 1, wherein the composition is preservative free.

7. The rehydrating composition as claimed in claim 1, wherein the composition has a pH in the range of 4.20 to 5.0 and the osmolarity is 245 mOsmol/L.

8. The rehydrating composition as claimed in claim 1, wherein the composition is a ready to drink (RTD) composition.

9. The rehydrating composition as claimed in claim 1, wherein the composition is packaged in a six layered tetra pack.

10. The rehydrating composition as claimed in claim 9, wherein the amount of the electrolyte, carbohydrates, zinc salts and post-biotics in the tetrapack is sufficient to prepare a single serving of oral rehydration solution having a volume of from 200 ml to 1 L.

11. The rehydrating composition as claimed in claim 1, wherein the composition is prepared by a process comprising combining water, the oral rehydrating solution, the carbohydrate, the zinc salt, and the post biotic.

12. A method of preventing diarrhea and restoring gut microbial flora comprising administering to a subject in need the rehydrating composition as claimed in claim 1.

13. A method of treating diarrhea and restoring gut microbial flora comprising administering to a subject in need the rehydrating composition as claimed in claim 1.

14. A rehydrating composition comprising:
i. an oral rehydrating solution (ORS) containing at least one electrolytes comprising sodium chloride in an amount of 26 g/100 ml, potassium chloride in an amount of 0.15 g/100 ml, and sodium citrate in an amount of 0.29 gm/100 ml;
ii. an anhydrous dextrose in an amount of 1.35 gm/100 ml;
iii zinc sulfate monohydrate comprising zinc in an amount of 4 mg/100 ml; and
iv. a postbiotic, wherein the postbiotic comprises heat killed Lactobacillus plantarum L-137 (HK L-137) in an amount of 25 mg/100 ml.
